(19) United States
(12) Patent Application Publication
(10) Pub. No.: US 2019/0247501 A1
(43) Pub. Date: Aug. 15, 2019

(54) MONOCLONAL ANTIBODY COCKTAILS FOR TREATMENT OF EBOLA INFECTIONS

(71) Applicant: MAPP BIOPHARMACEUTICAL, INC., SAN DIEGO, CA (US)

(72) Inventors: Andrew Hiatt, Hampton, VA (US); Larry Zeitlin, San Diego, CA (US); Kevin Whaley, Del Mar, CA (US); Michael Pauly, Del Mar, CA (US)

(21) Appl. No.: 16/370,801

(22) Filed: Mar. 29, 2019

Related U.S. Application Data
(62) Division of application No. 14/706,910, filed on May 7, 2015, now abandoned.

(51) Int. Cl. A61K 39/42; C07K 16/10

(52) U.S. Cl. CPC A61K 39/42; A61K 2039/507; C07K 2317/24; C07K 16/10

(57) ABSTRACT

Antibody variants originating from the monoclonal antibody 13C6, and wherein the N-glycosylation site within the constant region of the heavy chain contains a glycan that is either wild-type or largely devoid of fucose residues, will bind Ebola virus glycoprotein and provide surprising efficacy in treating animals or humans infected with Ebola virus when used in combination with one or more additional anti-Ebola mAbs. Such antibody cocktails are vastly superior to other known monoclonal antibodies or monoclonal antibody combinations in treating animals and humans infected with the Ebola virus.

### CROSS-REFERENCE TO RELATED APPLICATIONS

This application is a divisional application of U.S. patent application Ser. No. 14/706,910, filed May 7, 2015.

### BACKGROUND OF THE INVENTION

Ebola viruses are highly pathogenic and virulent viruses causing rapidly fatal hemorrhagic fever in humans. Cocktails of antibodies comprising two or more mAbs have been found to be more effective in treating infections with the Ebola virus than any individual mAb used alone (1-4). Antibody sequences that enable and optimize the mAb cocktails for treatment of Ebola are disclosed.

### SUMMARY OF THE INVENTION

We have surprisingly found that murine or humanized antibodies, wherein the CDRs originate from mouse monoclonal antibody 13C6 and the framework and other portions of the antibodies are of murine origin or originate from human germ line, and wherein an N-glycosylation site within the constant region of the heavy chain contains a glycan that is either wild-type or largely devoid of fucose residues, will bind Ebola virus glycoprotein and provide surprisingly excellent efficacy in treating animals or humans infected with Ebola virus when used in combination with one or more additional anti-Ebola mAb. Thus, we have a reasonable basis for believing that antibodies of this specificity offer the opportunity to treat, both prophylactically and therapeutically, conditions in humans that are associated with Ebola virus infection including haemorrhage, multi-organ failure and a shock-like syndrome.

Surprisingly, we have discovered that combinations of monoclonal antibodies comprising such a monoclonal antibody 13C6 as well as additional monoclonal antibodies specific to the Ebola glycoprotein are vastly superior to other known monoclonal antibodies or monoclonal antibody combinations in treating animals and humans infected with the Ebola virus.

According to a first aspect of the invention, there is provided a monoclonal antibody variable region comprising an amino acid sequence deduced from the heavy chain amino acid sequence of the 13C6 monoclonal antibody SEQ ID NO: 1 and the light chain variable region amino acid sequence SEQ ID NO: 2 as well as variants of these sequence that improve the effectiveness, stability, and solubility of the 13C6 antibody.

According to a second aspect of the invention, there is provided a method of preparing a chimeric antibody comprising: providing an expression vector comprising a nucleic acid molecule encoding a constant region domain of a human light chain or heavy chain genetically linked to a nucleic acid encoding a light chain variable region selected from the group consisting of the 13C6 heavy and light chains and variants of those sequences; expressing the expression vector in a suitable host; and recovering the chimeric antibody from said host.

According to a third aspect of the invention, there is provided a method of preparing recombinant antibodies comprising:

providing a nucleotide sequence selected from the group consisting of the 13C6 heavy chain nucleotide sequence SEQ ID NO: 3 and the light chain nucleotide sequence SEQ ID NO: 4 as well as variants of these sequence that improve the effectiveness, stability, and solubility of the 13C6 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence;

providing a nucleotide sequence selected from the group consisting of the 2G4 heavy chain nucleotide sequence SEQ ID NO: 5 and the light chain sequence SEQ ID NO: 6 as well as variants of these sequence that improve the effectiveness, stability, and solubility of the 2G4 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence; and

providing a nucleotide sequence selected from the group consisting of the 4G7 heavy chain nucleotide sequence SEQ ID NO: land the light chain sequence SEQ ID NO: 8 as well as variants of these sequence that improve the effectiveness, stability, and solubility of the 4G7 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence.

Thus, it is one embodiment of the present invention to provide a composition for the treatment of Ebola, the composition comprising: a therapeutically effective combination of i.) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, ii.) a second monoclonal antibody comprising a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof, and iii.) a third monoclonal antibody comprising a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

Such an embodiment may further comprise a pharmaceutically acceptable excipient or carrier.

Alternately, such an embodiment may be a composition wherein at least one of the first, second, and third monoclonal antibodies comprise a predominantly single glycoform.

It is yet another embodiment of the present invention to provide such a composition wherein the predominantly single glycoform comprises the GnGn glycan, galactosylated glycans, or sialylated glycans.

It is still another embodiment of the present invention to provide such a composition wherein the predominantly single glycoform comprises less than 5% fucose or xylose.

It is a second embodiment of the present invention to provide a composition for the treatment of Ebola, the composition comprising: a therapeutically effective combination of i.) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, and ii.) a second monoclonal antibody that binds the Ebola glycoprotein; iii.) wherein administration of the composition to patients five days following infection with the Ebola virus results in at least a 70% survival rate.

It is another embodiment of the present invention to provide such a composition, wherein the second monoclonal antibody comprises a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof.

It is still another embodiment of the present invention to provide such a composition, wherein the second monoclonal antibody comprises a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

It is yet another embodiment of the present invention to provide such a composition, wherein the patient is a human.

It is still another embodiment of the present invention to provide such a composition and further comprising: a pharmaceutically acceptable excipient or carrier.

It is yet another embodiment of the present invention to provide such a composition, wherein at least one of the first and second monoclonal antibodies comprise a predominantly single glycoform.

It is still another embodiment of the present invention to provide such a composition wherein the predominantly single glycoform comprises the GnGn glycan, galactosylated glycans, or sialylated glycans.

It is yet another embodiment of the present invention to provide such a composition wherein the predominantly single glycoform comprises less than 5% fucose or xylose.

It is a third embodiment of the present invention to provide a method for the treatment of Ebola infection in a patient, the method comprising: i.) identifying a patient in need of Ebola treatment; and ii.) administering to the patient a therapeutically effective amount of a composition comprising a combination of: a) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, b) a second monoclonal antibody comprising a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof, and c) a third monoclonal antibody comprising a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

It is another embodiment of the present invention to provide such a method, wherein the patient is a human.

It is yet another embodiment of the present invention to provide such a method, wherein the therapeutically effective composition further comprises a pharmaceutically acceptable excipient or carrier.

### BRIEF DESCRIPTION OF THE DRAWINGS

FIG. 1: A graph showing post-exposure protection of Ebola Virus infected nonhuman primates with ZMAPP.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned above and hereunder are incorporated herein by reference.

### Definitions

As used herein, “neutralizing antibody” refers to an antibody, for example, a monoclonal antibody (mAb), capable of disrupting a formed viral particle or inhibiting formation of a viral particle or prevention of binding to or infection of mammalian cells by a viral particle.

As used herein, “diagnostic antibody” or “detection antibody” or “detecting antibody” refers to an antibody, for example, a monoclonal antibody, capable of detecting the presence of an antigenic target within a sample. As will be appreciated by one of skill in the art, such diagnostic antibodies preferably have high specificity for their antigenic target.

As used herein, “humanized antibodies” refer to antibodies with reduced immunogenicity in humans.

As used herein, “chimeric antibodies” refer to antibodies with reduced immunogenicity in humans built by genetically linking a non-human variable region to human constant domains.

As used herein, the word “treat” includes therapeutic treatment, where a condition to be treated is already known to be present and prophylaxis—i.e., prevention of, or amelioration of, the possible future onset of a condition.

As used herein, a “therapeutically effective” treatment refers a treatment that is capable of producing a desired effect. Such effects include, but are not limited to, enhanced survival, reduction in presence or severity of symptoms, reduced time to recovery, and prevention of initial infection.

By “antibody” is meant a monoclonal antibody (mAb) per se, or an immunologically effective fragment thereof, such as an Fab, Fab′, or F(ab′)2 fragment thereof. In some contexts, herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, it is included within the term “antibody.” Also included within the definition “antibody” are single chain forms. Preferably, but not necessarily, the antibodies useful in the invention are produced recombinantly. Antibodies may or may not be glycosylated, though glycosylated. Antibodies are preferred. In a further preferred embodiment, the glycosylated antibodies contain glycans that are largely devoid of fucose. In another preferred embodiment, the glycosylated antibodies contain glycans that are galactosylated. In yet another preferred embodiment, the galactosylated antibodies contain glycans that are sialylated. Antibodies are properly cross-linked via disulfide bonds, as is well known.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within each isotype, there may be subtypes, such as IgG₁, IgG₂, IgG₃, IgG₄, etc. Within light and heavy chains, the variable and constant regions are joined by a “J” region of about 12 or more amino acids, with the heavy chain also including a “D” region of about 3 or more amino acids. The particular identity of constant region, the isotype, or subtype does not impact the present invention. The variable regions of each light/heavy chain pair form the antibody binding site.

Thus, an intact antibody has two binding sites. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat “Sequences of Proteins of Immunological Interest” National Institutes of Health, Bethesda, Md. 1987 and 1991; Chothia, et al., J. Mol. Biol. 196:901-917 (1987); Chothia, et al., Nature 342:878-883 (1989)].

By “humanized antibody” is meant an antibody that is composed partially or fully of amino acid sequences derived from a human antibody germline by altering the sequence of an antibody having non-human complementarity determining regions (CDR). A humanized immunoglobulin does not encompass a chimeric antibody, having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms. Humanized antibodies have at least three potential advantages over non-human and chimeric antibodies for use in human therapy:

1) Because the effector portion is human, it may interact better with the other parts of the human immune system (e.g., destroy the target cells more efficiently by complement-dependent cytotoxicity (CDC) or antibody-dependent cellular cytotoxicity (ADCC)).

2) The human immune system should not recognize the framework or C region of the humanized antibody as foreign, and therefore the antibody response against such an injected antibody should be less than against a totally foreign non-human antibody or a partially foreign chimeric antibody.

3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

The design of humanized immunoglobulins may be carried out as follows. As to the human framework region, a framework or variable region amino acid sequence of a CDR-providing non-human immunoglobulin is compared with corresponding sequences in a human immunoglobulin variable region sequence collection, and a sequence having a high percentage of identical amino acids is selected. When an amino acid falls under the following category, the framework amino acid of a human immunoglobulin to be used (acceptor immunoglobulin) is replaced by a framework amino acid from a CDR-providing non-human immunoglobulin (donor immunoglobulin):

(a) the amino acid in the human framework region of the acceptor immunoglobulin is unusual for human immunoglobulin at that position, whereas the corresponding amino acid in the donor immunoglobulin is typical for human immunoglobulin at that position; (b) the position of the amino acid is immediately adjacent to one of the CDRs; or (c) any side chain atom of a framework amino acid is within about 5-6 angstroms (center-to-center) of any atom of a CDR amino acid in a three dimensional immunoglobulin model [Queen, et al, Proc. Natl Acad. Sci. USA 86:10029-10033 (1989), and Co, et al., Proc. Natl. Acad. Sci. USA 88, 2869 (1991)]. When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

### The 13C6 mAb is an Essential Component of Antibody Cocktails for Ebola.

A variety of mAbs are available to create cocktails that are effective in neutralizing the Ebola virus, as has been described (1-4). Complete survival of guinea pigs or non-human primates after Ebola virus infection requires a cocktail of mAbs that includes 13C6 (3).

The CDRs of murine 13C6 have the following amino acid sequences:

light chain CDR1: SEQ ID NO: 9

light chain CDR2: SEQ ID NO: 10

light chain CDR3: SEQ ID NO: 11

heavy chain CDR1: SEQ ID NO: 12

heavy chain CDR2: SEQ ID NO: 13

heavy chain CDR3: SEQ ID NO: 14

Described herein are the 13C6 mAb and a number of variants of the 13C6 mAb that are effective in treating animals and human individuals infected with Ebola virus. Treatment is best accomplished by adding 13C6 to other anti-Ebola mAbs to create a cocktail of two or more mAbs. We have surprisingly found that other anti-Ebola mAbs are not as effective, either alone or in combination, as a cocktail containing 13C6. These cocktails can be tested in non-human primates infected with Ebola virus as described below.

These 13C6 antibodies and variants also appear to have high affinity and avidity to Ebola glycoproteins, which means that they could be used as highly sensitive diagnostic tools.

Humanized variants of 13C6 can include but are not limited to heavy chain FR variants

FR1: SEQ ID NO: 15;

FR2: SEQ ID NO: 16;

FR3: SEQ ID NO: 17;

and light chain FR variants

FR1: SEQ ID NO: 18;

FR2: SEQ ID NO: 19;

FR3: SEQ ID NO: 20;

or any other variant that minimizes the immunogenicity of the antibody in humans and retains antigen binding.

One or more of the sequences described herein comprising or encoding the 13C6 antibody can be subjected to humanization techniques or converted into chimeric human molecules for generating a variant antibody which has reduced immunogenicity in humans. Humanization techniques are well known in the art—see for example U.S. Pat. Nos. 6,309,636 and 6,407,213 which are incorporated herein by reference specifically for their disclosure on humanization techniques. Chimerics are also well known, see for example U.S. Pat. Nos. 6,461,824, 6,204,023, 6,020,153 and 6,120,767 which are similarly incorporated herein by reference. Such techniques can also be applied to antibodies other than 13C6, such as those described herein, to achieve predictable results.

In one embodiment of the invention, chimeric antibodies are formed by preparing an expression vector which comprises a nucleic acid encoding a constant region domain of a human light or heavy chain genetically linked to a nucleic acid encoding a light chain variable region selected from the group consisting of 13C6 and its variants disclosed herein.

Additional variants of 13C6 include but are not limited to mutations in FRs that improve the stability, solubility, and production. These mutations include but are not limited to the heavy chain sequences of SEQ ID NOs: 21-23.

Additional mutations include but are not limited to the light chain sequences of SEQ ID NOs: 24-25.

The heavy chain mutations can be combined with any of the light chain mutations to achieve the desired effect on expression, stability, or solubility when introduced into a host organism. In a preferred embodiment, the host organism for the production of wild-type and mutated sequences of 13C6 is Nicotiana benthamiana.

In another embodiment of the invention, there are provided recombinant antibodies comprising at least one modified variable region, said region selected from the group consisting of 13C6 and its variants in which at least one but fewer than about 30 of the amino acid residues of said variable region has been changed or deleted without disrupting antigen binding.

In yet other embodiments, immunoreactive fragments of any of the above-described monoclonal antibodies, chimeric antibodies or humanized antibodies are prepared using means known in the art, for example, by preparing nested deletions using enzymatic degradation or convenient restriction enzymes.

It is of note that in all embodiments describing preparation of humanized antibodies, chimeric antibodies or immunoreactive fragments of monoclonal antibodies, these antibodies are screened to ensure that antigen binding has not been disrupted. This may be accomplished by any of a variety of means known in the art, but one convenient method would involve use of a phage display library. As will be appreciated by one of skill in the art, as used herein, ‘immunoreactive fragment’ refers in this context to an antibody fragment reduced in length compared to the wild-type or parent antibody which retains an acceptable degree or percentage of binding activity to the target antigen. As will be appreciated by one of skill in the art, what is an acceptable degree will depend on the intended use.

It is of note that as discussed herein, any of the described antibodies or humanized variants thereof may be formulated into a pharmaceutical treatment for providing passive immunity for individuals suspected of or at risk of developing hemorrhagic fever comprising a therapeutically effective amount of said antibodies. The pharmaceutical preparation may include a suitable excipient or carrier. See, for example, Remington: The Science and Practice of Pharmacy, 1995, Gennaro ed. As will be apparent to one knowledgeable in the art, the total dosage will vary according to the weight, health and circumstances of the individual as well as the efficacy of the antibodies.

In another embodiment of the invention, there are provided glycoengineered variants of 13C6 and other monoclonal antibodies that contain predominantly a single glycoform. These glycans can be GnGn (GlcNAc₂-Man₃-GlcNAc₂), mono- or di-galactosylated (Gal_{(1/2)}-GlcNAc₂-Man₃-GlcNAc₂), mono- or di-sialylated (NaNa_{(1,2)}-Gal_{(1/2)}-GlcNAc₂-Man₃-GlcNAc₂) containing little or no fucose or xylose. A predominantly single glycoform is any glycoform that represents more than half (e.g. greater than 50%, 60%, 70%, 80%, 90%, 95%, 96%, 97%, 98%, 99%) of all glycoforms present in the antibody solution.

The RAMP system has been used for glycoengineering of antibodies, antibody fragments, idiotype vaccines, enzymes, and cytokines. Dozens of antibodies have been produced in the RAMP system by Mapp (5, 6) and others (7, 8). These have predominantly been IgGs but other isotypes, including IgM (9, 10), have been glycoengineered. Glycoengineering has also been extended to human enzymes in the RAMP system (11, 12). Since the RAMP system has a rapid turn-around time from Agrobacterium infection to harvest and purification (13) patient specific idiotype vaccines have been used in clinical trials for non-Hodgkins lymphoma (7).

For glycoengineering, recombinant Agrobacterium containing a 13C6 mAb cDNA is used for infection of N. benthamiana in combination with the appropriate glycosylation Agrobacteria to produce the desired glycan profile. For wild-type glycans (i.e. native, plant-produced glycosylation) wild-type N. benthamiana is inoculated with only the Agrobacterium containing the anti-M2e cDNA. For the GnGn glycan, the same Agrobacterium is used to inoculate plants that contain little or no fucosyl or xylosyl transfrases (ΔXF plants). For galactosylated glycans, ΔXF plants are inoculated with the Agrobacterium containing the 13C6 cDNA as well as an Agrobacterium containing the cDNA for β-1,4-galactosyltransferase expression contained on a binary Agrobacterium vector to avoid recombination with the TMV and PVX vectors (14). For sialylated glycans, six additional genes are introduced in binary vectors to reconstitute the mammalian sialic acid biosynthetic pathway. The genes are UDP-N-acetylglucosamine 2-epimerase/N-acetylmannosamine kinase, N-acetylneuraminic acid phosphate synthase, CMP-N-acetylneuraminic acid synthetase, CMP-NeuAc transporter, β-1,4-galactosylatransferase, and α2,6-sialyltransferase (14).

Glycanalysis of glycoengineered mAbs involved release of N-linked glycans by digestion with N-glycosidase F (PNGase F), and subsequent derivatization of the free glycan is achieved with anthranilic acid (2-AA). The 2-AA-derivatized oligosaccharide is separated from any excess reagent via normal-phase HPLC. The column is calibrated with 2-AA-labeled glucose homopolymers and glycan standards. The test samples and 2-AA-labeled glycan standards are detected fluorometrically. Glycoforms are assigned either by comparing their glucose unit (GU) values with those of the 2-AA-labeled glycan standards or by comparing with the theoretical GU values (15). Confirmation of glycan structure was accomplished with LC/MS.

While the RAMP system is an effective method of producing various glycoengineered and wild-type mABs, it will be recognized that other expression systems may be used to accomplish the same result. For example, mammalian cell lines (such as CHO or NSO cells [Davies, J., Jiang, L., Pan, L. 1, LaBarre, M. J., Anderson, D., and Reff, M. 2001. Expression of GnTIII in a recombinant anti-CD20 CHO production cell line: Expression of antibodies with altered glycoforms leads to an increase in ADCC through higher affinity for FCyRIII. Biotechnol Bioeng 74:288-294]), yeast cells (such as Pichia pastoris [Gerngross T. Production of complex human glycoproteins in yeast. Adv Exp Med Biol. 2005; 564]) and bacterial cells (such as E. coli) have been used produce such mABs.

TABLE 1
Glycananlysis of 2G4, 13C6FR1, and 4G7 antibodies produced using the RAMP system.

| | FLR | | | | | | |
|---|---|---|---|---|---|---|---|
| | RT | FLR Peak Area | | | | % Abundance | |
| Isoforms^{a} | (min) | C2G4 | C13C6FR1 | C4G7 | C2G4 | C13C6FR1 | C4G7 |
| unknown1 | 14.5 | 18864 | 11275 | 17640 | 0.8 | N/A | 0.6 |
| unknown2 | 15.8 | 20601 | 25759 | 28937 | 0.9 | 0.9 | 1.0 |
| unknown3 | 16.1 | 12637 | 15906 | 17746 | 0.6 | 0.5 | 0.6 |
| G0-GlcNAc | 17.7 | 27255 | 31183 | 20977 | 1.2 | 1.1 | 0.8 |
| G0 | 20.5 | 1988075 | 2358378 | 2584314 | 88.4 | 80.8 | 92.6 |
| G0-GlcNAc + Man | 21.9 | 17977 | 22786 | 10420 | 0.8 | 0.8 | N/A |
| Man5 | 23.4 | 31534 | 44372 | 11974 | 1.4 | 1.5 | N/A |
| G1(a) | 24.0 | 24183 | 11683 | 3936 | 1.1 | 0.4 | 0.1 |
| G1(b) | 24.4 | 25493 | 15866 | 12213 | 1.1 | 0.5 | N/A |
| G0-GlcNAc + 2Man | 25.6 | 21026 | 32958 | 4934 | 0.9 | 1.1 | N/A |
| Man6 | 27.2 | 5941 | 20789 | 1894 | N/A | 0.7 | N/A |
| Man7 | 30.6 | 17435 | 77267 | 13758 | 0.8 | 2.6 | 0.5 |
| Man8 | 33.9 | 22831 | 162345 | 37658 | 1.0 | 5.6 | 1.3 |
| Man9 | 36.3 | 15376 | 88381 | 25573 | 0.7 | 3.0 | 0.9 |

^{a}Only detected glycans with FLR peak area ≥ 0.5% relative to the most abundant glycan (G0) are reported in this table.

As illustrated in Table 1, the RAMP system is effective for producing monoclonal antibodies that have little or no fucose or xylose (for example less than 5% or less than 1% fucose or xylose). Isoforms containing fucose, xylose, or both could only be represented in the three “unknown” categories of Table 1.

While the preferred embodiments of the invention have been described above, it will be recognized and understood that various modifications may be made therein, and the appended claims are intended to cover all such modifications which may fall within the spirit and scope of the invention.

### 13C6 for Ebola Treatment

Without an approved vaccine or treatment, Ebola outbreak management has been limited to palliative care and barrier methods to prevent transmission. These approaches, however, have yet to end the 2014 outbreak of Ebola after its prolonged presence in West Africa. Here we show that a combination of monoclonal antibodies (ZMAPP), optimized from two previous antibody cocktails, is able to rescue 100% of rhesus macaques when treatment is initiated up to 5 days post-challenge. High fever, viremia, and abnormalities in blood count and chemistry were evident in many animals before ZMAPP intervention. Advanced disease, as indicated by elevated liver enzymes, mucosal hemorrhages and generalized petechia could be reversed, leading to full recovery. ELISA and neutralizing antibody assays indicate that ZMAPP is cross-reactive with the Guinean variant of Ebola. ZMAPP currently exceeds all previous descriptions of efficacy with other therapeutics, and results warrant further development of this cocktail for clinical use.

Ebola virus (EBOV) infections cause severe illness in humans, and after an incubation period of 3 to 21 days, patients initially present with general flu-like symptoms before a rapid progression to advanced disease characterized by hemorrhage, multi-organ failure and a shock-like syndrome (16). In the spring of 2014, a new EBOV variant emerged in the West African country of Guinea (17), an area in which EBOV has not been previously reported. Despite a sustained international response from local and international authorities including the Ministry of Health (MOH), World Health Organization (WHO) and Médecins Sans Frontières (MSF) since March 2014, the outbreak has yet to be brought to an end after five months. As of 15 Aug. 2014, there are 2127 total cases and 1145 deaths spanning Guinea, Sierra Leone, Liberia and Nigeria (18). So far, this outbreak has set the record for the largest number of cases and fatalities, in addition to geographical spread(19). Controlling an EBOV outbreak of this magnitude has proven to be a challenge and the outbreak is predicted to last for at least several more months (20). In the absence of licensed vaccines and therapeutics against EBOV, there is little that can be done for infected patients outside of supportive care, which includes fluid replenishment, administration of antivirals, and management of secondary symptoms (21) (22). With overburdened personnel, and strained local and international resources, experimental treatment options cannot be considered for compassionate use in an orderly fashion at the moment. However, moving promising strategies forward through the regulatory process of clinical development has never been more urgent.

Over the past decade, several experimental strategies have shown promise in treating EBOV-challenged nonhuman primates (NHPs) after infection. These include recombinant human activated protein C (rhAPC) (23), recombinant nematode anticoagulant protein c2 (rNAPc2) (24), small interfering RNA (siRNA) (25), positively-charged phosphorodiamidate morpholino oligomers (PMOplus) (26), the vesicular stomatitis virus vaccine (VSVΔG-EBOVGP)(27), as well as the monoclonal antibody (mAb) cocktails MB-003 (consisting of human or human-mouse chimeric mAbs c13C6, h13F6 and c6D8) (28) and ZMAb (consisting of murine mAbs m1H3, m2G4 and m4G7) (29) (U.S. Pat. No. 8,513,391). Of these, only the antibody-based candidates have demonstrated substantial benefits in NHPs when administered greater than 24 hours past EBOV exposure. Follow-up studies have shown that MB-003 is partially efficacious when administered therapeutically after the detection of two disease “triggers” (30), and ZMAb combined with an adenovirus-based adjuvant provides full protection in rhesus macaques when given up to 72 hours after infection (31).

Our objective was to develop a therapeutic superior to both MB-003 and ZMAb, which could be utilized for outbreak patients, primary health-care providers, as well as high-containment laboratory workers in the future. The study aimed to first identify an optimized antibody combination derived from MB-003 and ZMAb components, before determining the therapeutic limit of this mAb cocktail in a subsequent experiment. In order to extend the antibody half-life in humans and to facilitate clinical acceptance, the individual murine antibodies in ZMAb were first chimerized with human constant regions (cZMAb). The cZMAb components were then produced in Nicotiana benthamiana (32), using the large-scale, cGMP-compatible Rapid Antibody Manufacturing Platform (RAMP) and magnICON vectors that currently also manufactures the individual components of cocktail MB-003, before efficacy testing in animals.

### EXAMPLES

### Materials and Methods

### Ethics Statement

The guinea pig experiment, in addition to the second and third NHP study (ZMapp1, ZMapp2 and ZMAPP) were performed at the National Microbiology Laboratory (NML) as described on Animal use document (AUD) #H-13-003, and has been approved by the Animal Care Committee (ACC) at the Canadian Science Center for Human and Animal Health (CSCHAH), in accordance with the guidelines outlined by the Canadian Council on Animal Care (CCAC). The first study with MB-003 in NHPs was performed at United States Army Medical Research Institute of Infectious Diseases (USAMRIID) under an Institutional Animal Care and Use Committee (IACUC) approved protocol in compliance with the Animal Welfare Act, Public Health Service Policy, and other federal statutes and regulations relating to animals and experiments involving animals. The facility where this research was conducted in accredited by The Association for Assessment and Accreditation of Laboratory Animal Care International and adheres to principles stated in the 8^{th} edition of the Guide for the Care and Use of Laboratory Animals, National Research Council 2011.

### mAb Production

The large-scale production of mAb cocktails cZMAb, MB-003, ZMapp1, ZMapp2 and ZMAPP in addition to control mAb 4E10 (anti-HIV) from N. benthamiana under GMP conditions was done by Kentucky BioProcessing (Owensboro, Ky.) as described previously (28) (30) (33). The large-scale production of m4G7 was performed by the Biotechnology Research Institute (Montreal, QC) using a previously described protocol(31).

### Viruses

The challenge virus used in NHPs was Ebola virus H. sapiens-tc/COD/1995/Kikwit-9510621 (EBOV-K) (order Mononegavirales, family Filoviridae, species Zaire ebolavirus; GenBank accession #AY354458)(34). Passage three from the original stock was used for the studies at the NML and passage four was used for the study performed at USAMRIID (the NHP study with the individual MB-003 mAbs). Sequencing of 112 clones from the passage three stock virus revealed that the population ratio of 7U:8U in the EBOV GP editing site was 80:20; sequencing for the passage four stock virus was not performed, and therefore the ratio of 7U:8U in the editing site was unknown. The virus used in guinea pig studies was guinea pig-adapted EBOV, Ebola virus VECTOR/C. porcellus-lab/COD/1976/Mayinga-GPA (EBOV-M-GPA) (order Mononegavirales, family Filoviridae, species Zaire ebolavirus; Genbank accession number AF272001.1) (35). The Guinean variant used in IgG ELISA and neutralizing antibody assays was Ebola virus H. sapiens-tc/GIN/2014/Gueckedou-C05 (EBOV-G) (order Mononegavirales, family Filoviridae, species Zaire ebolavirus; GenBank accession #KJ660348.1) (17).

### Animals

Outbred 6-8 week old female Hartley strain guinea pigs (Charles River) were used for these studies. Animals were infected IP with 1000×LD₅₀ of EBOV-M-GPA. The animals were then treated with one dose of ZMAb, MB-003, ZMapp1, ZMapp2, c13C6, h13F6 or c6D8 totaling 5 mg per guinea pig, and monitored every day for 28 days for survival, weight and clinical symptoms. This study was not blinded, and no animals were excluded from the analysis.

For the MB-003 study performed at USAMRIID, thirteen rhesus macaques (Macaca mulatta) were obtained from the USAMRIID primate holding facility, ranging from 5.1 to 10 kg. This study was not blinded, and no animals were excluded from the analysis. Animals were given standard monkey chow, primate treats, fruits, and vegetables for the duration of the study. All animals were challenged IM with a target dose of 1000 PFU. Treatment with either monoclonal antibody, MB-003 cocktail, or PBS was administered on 1, 4, and 7 dpi via saphenous intravenous infusion. Animals were monitored at least once daily for changes in health, diet, behavior, and appearance. Animals were sampled for chemical analysis, complete bloods counts and viremia on 0, 3, 5, 7, 10, 14, 21, and 28 dpi.

For the ZMapp1 and ZMapp2 study, fourteen male and female rhesus macaques (Macaca mulatta), ranging from 4.1 to 9.6 kg (4-8 years old) were purchased from Primgen (USA). This study was not blinded, and no animals were excluded from the analysis. Animals were assigned groups based on gender and weight. Animals were fed standard monkey chow, fruits, vegetables, and treats. Husbandry enrichment consisted of visual stimulation and commercial toys. All animals were challenged IM with a high dose of EBOV [backtiter: 4000×TCID₅₀ or 2512 PFU] at 0 dpi. Administration of the first treatment dose was initiated at 3 dpi, with identical doses at 6 and 9 dpi. Animals were scored daily for signs of disease, in addition to changes in food and water consumption. On designated treatment days in addition to 14, 21, and 27 dpi, the rectal temperature and clinical score were measured, and the following were sampled: blood for serum biochemistry and cell counts and viremia. This study was not blinded, and no animals were excluded from the analysis.

For the ZMAPP study, twenty-one male rhesus macaques, ranging from 2.5 to 3.5 kg (2 years-old) were purchased from Primgen (USA). This study was not blinded, and no animals were excluded from the analysis. Animals were assigned groups based on gender and weight. Animals were fed standard monkey chow, fruits, vegetables, and treats. Husbandry enrichment consisted of visual stimulation and commercial toys. All animals were challenged IM with EBOV [backtiter: 1000×TCID₅₀ or 628 PFU] at 0 dpi. Administration of the first treatment dose was initiated at 3, 4 or 5 dpi, with two additional identical doses spaced three days apart. Animals were scored daily for signs of disease, in addition to changes in food and water consumption. On designated treatment days in addition to 14, 21, and 28 dpi, the rectal temperature and clinical score were measured, and the following were sampled: blood for serum biochemistry and cell counts and viremia.

### Blood Counts and Blood Biochemistry

Complete blood counts were performed with the VetScan HM5 (Abaxis Veterinary Diagnostics). The following parameters were shown in the figures: levels of white blood cells (WBC), lymphocytes (LYM), percentage of lymphocytes (LYM %), levels of platelets (PLT), neutrophils (NEU) and percentage of neutrophils (NEU %). Blood biochemistry was performed with the VetScan VS2 (Abaxis Veterinary Diagnostics). The following parameters were shown in the figures: levels of alkaline phosphatase (ALP), alanine aminotransferase (ALT), blood urea nitrogen (BUN), creatinine (CRE), and total bilirubin (TBIL).

### Enzyme-Linked Immunosorbent Assays (ELISAs)

IgG ELISA with c13C6, c2G4 or c1H3 was performed as described previously(31) using gamma-irradiated EBOV-G and EBOV-K virions purified on a 20% sucrose cushion as the capture antigen in the ELISA. Each mAb was assayed in triplicate.

### Neutralizing Antibody Assays

Two-fold dilutions of c13C6, c2G4 or c1H3 ranging from 0.0156 to 2 mg were first incubated with 100 PFU of EBOV-G at room temperature for 1 hour with or without complement, transferred to Vero E6 cells and incubated at 37° C. for 1 hour, and then replaced with DMEM supplemented with 2% fetal bovine serum and scored for the presence of cytopathic effect (CPE) at 14 dpi. The lowest concentrations of mAbs demonstrating the absence of CPE were averaged and reported.

### EBOV Titration by TCID₅₀ and RT-qPCR

Titration of live EBOV was determined by adding 10-fold serial dilutions of whole blood to VeroE6 cells, with three replicates per dilution. The plates were scored for cytopathic effect at 14 dpi, and titers were calculated with the Reed and Muench method (36). Results were shown as median tissue culture infectious dose (TCID₅₀).

For titers measured by RT-qPCR, total RNA was extracted from whole blood with the QIAmp Viral RNA Mini Kit (Qiagen). EBOV was detected with the LightCycler 480 RNA Master Hydrolysis Probes (Roche) kit, with the RNA polymerase (nucleotides 16472 to 16538, AF086833) as the target gene. The reaction conditions were as follows: 63° C. for 3 min, 95° C. for 30 s, and cycling of 95° C. for 15 s, 60° C. for 30 s for 45 cycles on the ABI StepOnePlus. The lower detection limit for this assay is 86 genome equivalents/ml. The sequences of primers used were as follows: EBOVLF2 (CAGCCAGCAATTTCTTCCAT), EBOVLR2 (TTTCGGTTGCTGTTTCTGTG), and EBOVLP2FAM (FAM-ATCATTGGCGTACTGGAGGAGCAG-BHQ1).

### Sequence Alignment

Protein sequences for EBOV-K and EBOV-G surface glycoproteins were obtained from GenBank, accession numbers AGB56794.1 and AHX24667.1 respectively. The sequences were aligned using DNASTAR Lasergene 10 MEGAlign using the Clustal W algorithm.

### Statistical Analysis

For the guinea pig and nonhuman primate studies, each treatment group consisted of six animals. Assuming a significance threshold of 0.05, a sample size of six per group will give >80% power to detect a difference in survival proportions between the treatment (83% survival or higher) and the control group using a one-tailed Fisher's exact test.

Survival was compared using the log-rank test in GraphPad PRISM 5, differences in survival were considered significant when the p-value was less than 0.05. Antibody binding to EBOV-G and EBOV-K was compared by fitting the data to a 4-parameter logistic regression using GraphPad PRISM 5. The EC₅₀ were considered different if the 95% Confidence Intervals excluded each other. For all statistical analyses, the data conformed to the assumptions of the test used.

### Example 1. Selection of the Best mAb Combinations

Our efforts to down-select for an improved mAb cocktail comprising components of MB-003 and ZMAb began with the testing of individual MB-003 antibodies in guinea pigs and NHPs. In guinea pig studies, animals were given one dose of mAb c13C6, h13F6, or c6D8 individually (totaling 5 mg per animal) at 1 day post-infection (dpi) with 1000×LD₅₀ of guinea pig-adapted EBOV, Mayinga variant (EBOV-M-GPA). Survival and weight loss were monitored over 28 days. Treatment with c13C6 or h13F6 yielded 17% survival (1 of 6 animals) with a mean time to death of 8.4±1.7 and 10.2±1.8 days, respectively. The average weight loss for c13C6 or h13F6-treated animals was 9% and 21% (Table 2). In nonhuman primates, animals were given three doses of mAb c13C6, h13F6, or c6D8, beginning at 24 hours after challenge with the Kikwit variant of EBOV (EBOV-K)(34), and survival was monitored over 28 days. Only c13C6 treatment yielded any survivors, with 1 of 3 animals protected from EBOV challenge (Table 2), confirming in two separate animal models that c13C6 is the component that provides the highest level of protection in the MB-003 cocktail.

We then tested mAb c13C6 in combination with two of three mAbs from ZMAb in guinea pigs. The individual antibodies composing ZMAb were originally chosen for protection studies based on their in vivo protection of guinea pigs against EBOV-M-GPA(37), and all three possible combinations were tested: ZMapp1 (c13C6+c2G4+c4G7), ZMapp2 (c13C6+c1H3+c2G4) and ZMapp3 (c13C6+c1H3+c4G7), and compared to the originator cocktails ZMAb and MB-003. Three days after challenge with 1000×LD₅₀ of EBOV-M-GPA, the animals received a single combined dose of 5 mg of antibodies. This dosage is purposely given to elicit a suboptimal level of protection with the cZMAb and MB-003 cocktails, such that potential improvements with the optimized mAb combinations can be identified. Of the tested cocktails, ZMapp1 showed the best protection, with 4 of 6 survivors and less than 5% average weight loss (Table 2). ZMapp2 was next with 3 of 6 survivors and 8% average weight loss, and ZMapp3 protected 1 of 6 animals (Table 2). The level of protection afforded by ZMapp3 was not a statistically significant increase over cZMAb (p=0.224, log-rank test compared to ZMAb, χ²=1.479, df=1), and showed the same survival rate along with a similar average weight loss (Table 2). As a result, only ZMapp1 and ZMapp2 were carried forward to NHP studies.

TABLE 2
Efficacy of individual and combined monoclonal antibody
treatments in guinea pigs and nonhuman primates

| Treatment | | Mean time to | | | | P value, | |
|---|---|---|---|---|---|---|---|
| groups, time | Dose | death | No. | Survival | Weight | compared with | |
| of treatment | (mg) | (days ± s.d.) | survivors/total | (%) | loss (%) | cZMAb | MB-003 |
| Guinea pigs | | | | | | — | — |
| PBS, 3 dpi | N/A | 7.3 ± 0.5 | 0/4 | 0 | 9% | — | — |
| cZMAb, 3 dpi | 5 | 11.6 ± 1.8 | 1/6 | 17 | 7% | — | — |
| MB-003, 3 dpi | 5 | 8.2 ± 1.5 | 0/6 | 0 | 40% | — | — |
| ZMapp1, 3 dpi | 5 | 9.0 ± 0.0 | 4/6 | 67 | <5% | 0.190 | 0.0147 |
| ZMapp2, 3 dpi | 5 | 8.3 ± 0.6 | 3/6 | 50 | 8% | 0.634 | 0.0692 |
| ZMapp3, 3 dpi | 5 | 8.6 ± 1.1 | 1/6 | 17 | 9% | 0.224 | 0.411 |
| c13C6, 1 dpi | 5 | 8.4 ± 1.7 | 1/6 | 17 | 9% | — | — |
| h13F6, 1 dpi | 5 | 10.2 ± 1.8 | 1/6 | 17 | 21% | — | — |
| c6D8, 1 dpi | 5 | 10.5 ± 2.2 | 0/6 | 0 | 38% | — | — |
| Nonhuman | | | | | | | |
| primates | | | | | | | |
| PBS, 1 dpi | N/A | 8.4 ± 1.9 | 0/1 | 0 | | | |
| MB-003, 1 dpi | 50 | 14.0 ± 2.8 | 1/3 | 33 | | | |
| c13C6, 1 dpi | 50 | 9.0 ± 1.4 | 1/3 | 33 | | | |
| h13F6, 1 dpi | 50 | 9.0 ± 2.0 | 0/3 | 0 | | | |
| c6D8, 1 dpi | 50 | 9.7 ± 0.6 | 0/3 | 0 | | | |

### Example 2. Deciding Between ZMapp1 or ZMapp2 Using Non-Human Primates (NHPs)

Rhesus macaques were used to determine whether administration of ZMapp1 or ZMapp2 was superior to ZMAb and MB-003 in terms of extending the treatment window. The experiment consisted of six NHPs per group receiving three doses of ZMapp1 or ZMapp2 at 50 mg/kg intravenously (IV) at 3-day intervals, beginning 3 days after a lethal intramuscular (IM) challenge with 4000×TCID₅₀ (or 2512 PFU) of EBOV-K. Control animals were given phosphate-buffered saline (PBS) or mAb 4E10. Mock-treated animals succumbed to disease between 6-7 dpi with symptoms typical of EBOV, characterized by high clinical scores but no fever, in addition to viral titers up to ˜10⁸ and ˜10⁹ TCID₅₀ by the time of death.

All six ZMapp1 treated NHPs survived the challenge with mild signs of disease (p=0.0039, log-rank test, χ²=8.333, df=1), comparing to control animals. A fever was detected in all but one of the NHPs at 3 dpi, the start of the first ZMapp1 dose. Viremia was also detected beginning at 3 dpi by TCID₅₀ in all but one animal from blood sampled just before the administration of the treatment, and similar results were observed by RT-qPCR. The viremia decreased to undetectable levels by 21 dpi. EBOV shedding was not detected from oral, nasal and rectal swabs by RT-qPCR in any of the ZMapp1 treated animals.

TABLE 3
Clinical findings of EBOV-infected NHPs from 1 to 27 dpi

| | | Clinical findings | | | | | |
|---|---|---|---|---|---|---|---|
| Animal | | Body | | White blood | | | |
| ID | Treatment group | temp. | Rash | cells | Platelets | Biochemistry | Outcome |
| A1 | 50 mg kg⁻¹c13C6 + c2G4 + m4G7, | Fever (6, | | | Thrombocytopenia | ALT↑ (9, | Survived |
| | 4G7, 3 dpi | 9, 14 dpi) | | | (6, 9 dpi) | 14 dpi), | |
| | | | | | | TBIL↑ | |
| | | | | | | (9 dpi), | |
| | | | | | | PHOS↓ | |
| | | | | | | (6 dpi) | |
| A2 | 50 mg kg⁻¹c13C6 + c2G4 + m4G7, | Fever | | Leukocytosis | | CRE↓ | Survived |
| | 3 dpi | (3 dpi) | | (3 dpi) | | (14 dpi) | |
| A3 | 50 mg kg⁻¹c13C6 + c2G4 + m4G7, | Fever | | Leukocytosis | Thrombocytopenia | | Survived |
| | 3 dpi | (3 dpi) | | (3 dpi) | (6 dpi) | | |
| A4 | 50 mg kg⁻¹c13C6 + c2G4 + m4G7, | | | Leukocytopenia | Thrombocytopenia | | Survived |
| | 3 dpi | | | (9 dpi) | (3, 6, 14, | | |
| | | | | | 21, 27 dpi) | | |
| A5 | 50 mg kg⁻¹c13C6 + c2G4 + m4G7, | Fever (3, | | Leukocytopenia | Thrombocytopenia | | Survived |
| | 3 dpi | 6, 9 dpi) | | (9 dpi) | (3, 21 dpi) | | |
| A6 | 50 mg kg⁻¹c13C6 + c2G4 + m4G7, | Fever | | | | | Survived |
| | 3 dpi | (3 dpi) | | | | | |
| B1 | 50 mg kg⁻¹ ZMapp2, 3 dpi | Fever (3, | | Leukocytopenia | Thrombocytopenia | | Survived |
| | | 14, 21 dpi) | | (6, 14, | (6 dpi) | | |
| | | | | 21, 27 dpi) | | | |
| B2 | 50 mg kg⁻¹ ZMapp2, 3 dpi | Fever (3, | | | Thrombocytopenia | | Survived |
| | | 6 dpi) | | | (6, 9 dpi) | | |
| B3 | 50 mg kg⁻¹ ZMapp2, 3 dpi | Fever (3, | Severe | | Thrombocytopenia | ALT↑↑↑ | Died, |
| | | 6 dpi), | rash | | (6, 9 dpi) | (9 dpi), | 9 dpi |
| | | Hypothermia | (9 dpi) | | | TBIL↑↑ | |
| | | (9 dpi) | | | | (9 dpi), | |
| | | | | | | BUN↑↑↑ | |
| | | | | | | (9 dpi), | |
| | | | | | | CRE↑↑↑ | |
| | | | | | | (9 dpi), | |
| | | | | | | GLU↓↓ | |
| | | | | | | (9 dpi) | |
| B4 | 50 mg kg⁻¹ ZMapp2, 3 dpi | Fever (3, | | Leukocytopenia | Thrombocytopenia | | Survived |
| | | 6 dpi) | | (6 dpi) | (6, 27 dpi) | | |
| B5 | 50 mg kg⁻¹ ZMapp2, 3 dpi | Fever (3, | | Leukocytosis | Thrombocytopenia | | Survived |
| | | 6, 14, | | (3 dpi) | (3, 6 dpi) | | |
| | | 21 dpi) | | | | | |
| B6 | 50 mg kg⁻¹ ZMapp2, 3 dpi | Fever | | Leukocytosis | Thrombocytopenia | PHOS↓ | Survived. |
| | | (3 dpi) | | (3 dpi), | (6 dpi) | (3 dpi), | |
| | | | | Leukocytopenia | | CRE↓ | |
| | | | | (6, 9, 14, | | (27 dpi) | |
| | | | | 21, 27 dpi) | | | |
| C1 | PBS, 3 dpi | | Moderate | Leukocytosis | Thrombocytopenia | ALB↓ | Died, |
| | | | rash | (3 dpi) | (6, 7 dpi) | (7 dpi), | 7 dpi |
| | | | (6 dpi), | | | ALT↑ | |
| | | | Severe | | | (7 dpi), | |
| | | | rash | | | BUN↑ | |
| | | | (7 dpi) | | | (7 dpi) | |
| C2 | Control mAb, 3 dpi | | Severe | Leukocytopenia | Thrombocytopenia | ALP↑ | Died, |
| | | | rash | (6, 7 dpi) | (6, 7 dpi) | (3 dpi), | 6 dpi |
| | | | (6 dpi) | | | ALT↑↑↑ | |
| | | | | | | (6 dpi), | |
| | | | | | | BUN↑ | |
| | | | | | | (6 dpi), | |
| | | | | | | CRE↑↑↑ | |
| | | | | | | (6 dpi) | |

In Table 3, hypothermia was defined as below 35° C. Fever was defined as >1.0° C. higher than baseline. Mild rash was defined as focal areas of petechiae covering <10% of the skin, moderate rash as areas of petechiae covering 10 to 40% of the skin, and severe rash as areas of petechiae and/or ecchymosis covering >40% of the skin. Leukocytopenia and thrombocytopenia were defined as a >30% decrease in numbers of WBCs and platelets, respectively. Leukocytosis and thrombocytosis were defined as a twofold or greater increase in numbers of WBCs and platelets over baseline, where WBC count >11.000. ↑, two- to threefold increase; ↑↑, four- to fivefold increase; ↑↑↑, greater than fivefold increase; ↓, two- to threefold decrease; ↓↓, four- to fivefold decrease; ↓↓↓, greater than fivefold decrease. ALB, albumin; AMY, amylase; TBIL, total bilirubin; BUN, blood urea nitrogen; PHOS, phosphate; CRE, creatinine; GLU, glucose; GLOB, globulin.

For ZMapp2 treated animals, 5 of 6 NHPs survived with one NHP succumbing to disease at 9 dpi (p=0.0039, log-rank test, χ²=8.333, df=1, comparing to control animals). Surviving animals showed only mild signs of disease (Table 3). The moribund animal showed increased clinical scores, in addition to a drastic drop in body temperature shortly before death. All six ZMapp2 treated animals showed fever in addition to viremia at 3 dpi by TCID₅₀ and RT-qPCR. The administration of ZMapp2 at the reported concentrations was unable to effectively control viremia. Virus shedding was also detected from the oral and rectal swabs by RT-qPCR in the moribund NHP. Since ZMapp1 demonstrated superior protection to ZMapp2 in this survival study, ZMapp1 (now trademarked as ZMAPP by MappBio Pharmaceuticals) was carried forward to test the limits of protection conferred by this mAb cocktail in a subsequent investigation.

### Example 3. Post-Exposure Protection of EBOV-Infected Nonhuman Primates with ZMAPP

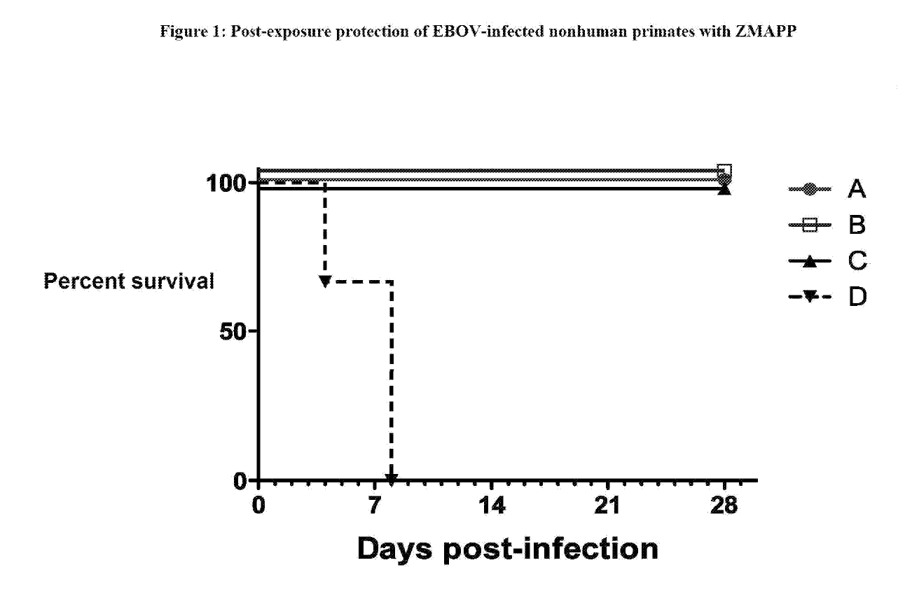

In this experiment, rhesus macaques were assigned into three treatment groups of six and a control group of three animals, with all treatment NHPs receiving three doses of ZMAPP (c13C6+c2G4+c4G7, 50 mg/kg per dose) spaced three days apart. After a lethal IM challenge with 1000×TCID₅₀ (or 628 PFU) of EBOV-K(34), we treated the animals with ZMAPP at 3, 6 and 9 dpi (Group A); 4, 7, and 10 dpi (Group B); or 5, 8 and 11 dpi (Group C). The control animals (Group D) were given mAb 4E10 as an IgG isotype control (n=1) or PBS (n=2) in place of ZMAPP starting at 4 dpi. All animals treated with ZMAPP survived the infection, whereas the three control NHPs (D1, D2 and D3) succumbed to EBOV-K infection at 4, 8 and 8 dpi, respectively (p=3.58E-5, log-rank test, χ²=23.25, df=3, comparing all groups) (FIG. 1). At the time ZMAPP treatment was initiated, fever, leukocytosis, thrombocytopenia and viremia could be detected in the majority of the animals. All animals presented with detectable abnormalities in blood counts and serum biochemistry during the course of the experiment.

Rhesus macaques (n=6 per ZMAPP treatment group, n=3 for controls) were challenged with EBOV-K, and 50 mg/kg of ZMAPP were administered beginning at 3 (Group A), 4 (Group B) or 5 (Group C) days after challenge. Non-specific IgG mAb or PBS was administered as a control (Group D) The Kaplan-Meier survival curves for each group is shown above.

TABLE 4
Clinical findings of EBOV-infected NHPs from 1 to 28 dpi

| | | Clinical findings | | | | | |
|---|---|---|---|---|---|---|---|
| Animal | | Body | | White blood | | | |
| ID | Treatment group | temperature | Rash | cells | Platelets | Biochemistry | Outcome |
| D1 | 50 mg kg⁻¹ZMapp, | Fever (3, 6, | | Leukocytosis | Thrombocytopenia | ALB↓ (14, | Survived |
| | 3 dpi | 14, 21 dpi) | | (3, 6, 21 dpi) | (3, 6, 9, 14, | 21 dpi), | |
| | | | | | 21 dpi) | ALP↓ (9, 14, | |
| | | | | | | 21, 28 dpi), | |
| | | | | | | AMY↓ | |
| | | | | | | (9 dpi), | |
| | | | | | | GLOB↑ (21, | |
| | | | | | | 28 dpi) | |
| D2 | 50 mg kg⁻¹ZMapp, | | | Leukocytopenia | Thrombocytopenia | PHOS↓ | Survived |
| | 3 dpi | | | (21, 28 dpi) | (28 dpi) | (9 dpi) | |
| D3 | 50 mg kg⁻¹ZMapp, | Fever | | Leukocytosis | Thrombocytopenia | ALT↓ (6 dpi) | Survived |
| | 3 dpi | (3 dpi) | | (3, 14 dpi) | (3, 21, 28 dpi) | | |
| D4 | 50 mg kg⁻¹ZMapp, | | | Leukocytopenia | Thrombocytopenia | ALT↓ | Survived |
| | 3 dpi | | | (14 dpi) | (14, 21 dpi) | (9 dpi), | |
| | | | | | | CRE↑ | |
| | | | | | | (14 dpi) | |
| D5 | 50 mg kg⁻¹ZMapp, | Fever | | Leukocytopenia | Thrombocytopenia | ALB↓ | Survived |
| | 3 dpi | (3 dpi) | | (21, 28 dpi) | (6, 9 dpi) | (9 dpi), | |
| | | | | | | BUN↓ (3, 6, | |
| | | | | | | 14, 21, | |
| | | | | | | 28 dpi) | |
| D6 | 50 mg kg⁻¹ZMapp, | | | | Thrombocytopenia | | Survived |
| | 3 dpi | | | | (6 dpi) | | |
| E1 | 50 mg kg¹ZMapp, | | | | Thrombocytopenia | AMY↓↓ (4, | Survived |
| | 4 dpi | | | | (4, 7, 21 dpi) | 21 dpi), | |
| | | | | | | AMY↓ (7, | |
| | | | | | | 10, 14 dpi), | |
| | | | | | | CRE↓ (21, | |
| | | | | | | 28 dpi) | |
| E2 | 50 mg kg⁻¹ZMapp, | Fever | | Leukocytosis | Thrombocytopenia | ALT ↓↓ | Survived |
| | 4 dpi | (4 dpi) | | (4, 10 dpi) | (4, 7, 10, 21 dpi) | (4 dpi), | |
| | | | | | | GLU↑ (4 dpi) | |
| E3 | 50 mg kg⁻¹ZMapp, | Fever | | Leukocytosis | Thrombocytopenia | CRE↓ | Survived |
| | 4 dpi | (4 dpi) | | (4, 10 dpi) | (7, 10, 14 dpi) | (14 dpi) | |
| E4 | 50 mg kg¹ZMapp, | | Severe | Leukocytosis | Thrombocytopenia | ALP↑ (7, 10, | Survived |
| | 4 dpi | | rash | (10, 14, 21, | (4, 7, 10, 14 dpi) | 14 dpi), ALT | |
| | | | (5, 6, | 28 dpi) | | ↑↑↑ (7 dpi), | |
| | | | 7, | | | ALT ↑↑ | |
| | | | 8 dpi), | | | (10 dpi), | |
| | | | Mild | | | AMY↓ (4, 7, | |
| | | | rash | | | 10 dpi), | |
| | | | (9 dpi) | | | TBIL↑↑↑ | |
| | | | | | | (7 dpi), | |
| | | | | | | TBIL↑ (10, | |
| | | | | | | 14 dpi), | |
| | | | | | | PHOS↓ (7, | |
| | | | | | | 10 dpi), K⁺↓ | |
| | | | | | | (4 dpi) | |
| E5 | 50 mg kg⁻¹ZMapp, | Fever | | Leukocytosis | Thrombocytopenia | ALT↑ | Survived |
| | 4 dpi | (7 dpi) | | (4 dpi) | (4, 7, 10, 14 dpi) | (7 dpi), | |
| | | | | | | AMY↓ (4, | |
| | | | | | | 7 dpi), | |
| | | | | | | PHOS↓ | |
| | | | | | | (10 dpi) | |
| E6 | 50 mg kg⁻¹ZMapp, | Fever | Mild | Leukocytosis | Thrombocytopenia | ALP↑ (7, | Survived |
| | 4 dpi | (4 dpi) | rash | (4, 10, 14 dpi) | (4, 7, 10, 14 dpi) | 10 dpi), ALT | |
| | | | (7, 8, | | | ↑↑↑ (7, 10, | |
| | | | 9 dpi) | | | 14 dpi), | |
| | | | | | | AMY↓ (7, | |
| | | | | | | 10 dpi), | |
| | | | | | | TBIL↑↑ | |
| | | | | | | (7 dpi), | |
| | | | | | | TBIL↑↑↑ | |
| | | | | | | (10 dpi), | |
| | | | | | | TBIL↑ | |
| | | | | | | (14 dpi), | |
| | | | | | | PHOS↓ | |
| | | | | | | (7 dpi), | |
| | | | | | | GLOB↑ | |
| | | | | | | (21 dpi) | |
| F1 | 50 mg kg⁻¹ZMapp, | | | Leukocytosis | Thrombocytopenia | AMY↓ | Survived |
| | 5 dpi | | | (11 dpi) | (3, 5, 8, 11 dpi) | (5 dpi), | |
| | | | | | | PHOS↓ | |
| | | | | | | (11 dpi), | |
| | | | | | | CRE↓ | |
| | | | | | | (28 dpi) | |
| F2 | 50 mg kg⁻¹ZMapp, | Fever (3, | Mild | Leukocytosis | Thrombocytopenia | PHOS↓ | Survived |
| | 5 dpi | 5 dpi) | rash | (3, 5, 11 dpi) | (3, 5, 8, 11, 14, | (11 dpi), | |
| | | | (8 dpi) | | 21 dpi) | CRE↓↓ | |
| | | | | | | (11 dpi) | |
| F3 | 50 mg kg⁻¹ZMapp, | | | Leukocytopenia | Thrombocytopenia | ALT↑ | Survived |
| | 5 dpi | | | (8 dpi), | (5, 8, 11, 21 dpi) | (8 dpi), | |
| | | | | Leukocytosis | | CRE↓↓ | |
| | | | | (3 dpi) | | (14 dpi) | |
| F4 | 50 mg kg⁻¹ZMapp, | Fever (3, | | Leukocytopenia | Thrombocytopenia | PHOS↓ | Survived |
| | 5 dpi | 5 dpi) | | (8 dpi) | (5, 8, 11, 28 dpi) | (8 dpi) | |
| F5 | 50 mg kg⁻¹ZMapp, | Fever | | Leukocytosis | Thrombocytopenia | PHOS↓ (5, | Survived |
| | 5 dpi | (3 dpi) | | (3, 11, 14 dpi) | (5, 8, 11 dpi) | 8 dpi), CRE↓ | |
| | | | | | | (8, 11, 21, | |
| | | | | | | 28 dpi) | |
| F6 | 50 mg kg⁻¹ZMapp, | Fever | | Leukocytopenia | Thrombocytopenia | PHOS↓ (5, 8, | Survived |
| | 5 dpi | (3 dpi) | | (8, 21, 28 dpi) | (8, 11,21 dpi) | 11 dpi), | |
| | | | | | | GLU↑ (5 dpi) | |
| G1 | PBS, 4 dpi | | Severe | Leukocytopenia | Thrombocytopenia | AMY↓ | Died, |
| | | | rash | (4 dpi) | (4 dpi) | (4 dpi) | 4 dpi |
| | | | (4 dpi) | | | | |
| G2 | Control mAb, | | Severe | Leukocytopenia | Thrombocytopenia | ALP↑ | Died, |
| | 4 dpi | | rash | (7, 8 dpi) | (4, 7, 8 dpi) | (8 dpi), | 8 dpi |
| | | | 8 dpi) | | | ALT↑ | |
| | | | | | | (7 dpi), ALT | |
| | | | | | | ↑↑↑ (8 dpi), | |
| | | | | | | CRE↑ (8 dpi) | |
| G3 | PBS, 4 dpi | Fever (4, | Severe | Leukocytopenia | Thrombocytopenia | ALP↑ | Died, |
| | | 8 dpi) | rash | (7, 8 dpi) | (4, 7, 8 dpi) | (8 dpi), | 8 dpi |
| | | | (8 dpi), | | | ALT↑ (7, | |
| | | | | | | 8 dpi), | |
| | | | | | | AMY↓ | |
| | | | | | | (7 dpi), AMY | |
| | | | | | | ↓↓ (8 dpi) | |
| | | | | | | TBIL↑ | |
| | | | | | | PHOS↓ | |
| | | | | | | (7 dpi) | |

In Table 4 hypothermia was defined as below 35° C. Fever was defined as >1.0° C. higher than baseline. Mild rash was defined as focal areas of petechiae covering <10% of the skin, moderate rash was defined as areas of petechiae covering 10 to 40% of the skin, and severe rash was defined as areas of petechiae and/or ecchymosis covering >40% of the skin. Leukocytopenia and thrombocytopenia were defined as a >30% decrease in the numbers of WBCs and platelets, respectively. Leukocytosis and thrombocytosis were defined as a twofold or greater increase in numbers of WBCs and platelets above baseline, where WBC counts are greater than 11.0. ↑, two- to threefold increase; ↑↑, four- to fivefold increase; ↑↑↑, greater than fivefold increase; ↓, two- to threefold decrease; ↓↓, four- to fivefold decrease; ↓↓↓, greater than fivefold decrease. ALB, albumin; ALP, alkaline phosphatase; ALT, alanine aminotransferase; AMY, amylase; TBIL, total bilirubin; BUN, blood urea nitrogen; PHOS, phosphate; CRE, creatinine; GLU, glucose; K⁺, potassium; GLOB, globulin.

In another set of experiments (Table 5) pairs of mAbs were used to treat non-human primates. The combination of 13C6 and 2G4 resulted in an equivalent survival rate compared to ZMAPP.

TABLE 5
Efficacy of pairs of mAbs for treatment
of non-human primates at 3 DPI.

| | | | Mean time | |
|---|---|---|---|---|
| | Surviving/ | Survival | to death | Weight |
| Treatment groups | Total animals | (%) | (days) ± SD | loss (%) |
| ZMapp-N (1:1:1) | 6/8 | 75% | 9.5 ± 2.1 | <5% |
| (n = 8) | | | | |
| ZMapp-CHO (1:1:1) | 4/6 | 67% | 13.5 ± 3.5 | <5% |
| (n = 6) | | | | |
| 13C6 − N + | 5/6 | 83% | 17 | <5% |
| (2G4 + 4G7) − | | | | |
| CHO (1:1:1) (n = 6) | | | | |
| (13C6 + 2G4) − N | 2/8 | 25% | 11.7 ± 2.9 | 13% |
| (1:1) (n = 8) | | | | |
| (13C6 + 2G4) − N | 6/8 | 75% | 9.5 ± 0.7 | <5% |
| (1:2) (n = 8) | | | | |
| (1H3 + 2G4 + 5D2) − | 2/6 | 33% | 15.5 ± 4.4 | 18% |
| CHO (1:1:1) (n = 6) | | | | |
| PBS (n = 4) | 0/4 | 0% | 7.5 ± 0.6 | 26% |
| ZMAb (1:1:1) (n = 4) | 1/4 | 25% | 8 ± 0 | 10% |
| 13C6 + 4G7 − N | 2/4 | 50% | 7.5 ± 0.7 | <5% |
| (1:1) (n = 4) | | | | |
| 13C6 + 4G7 − N | 1/4 | 25% | 9.3 ± 0.6 | <5% |
| (1:2) (n = 4) | | | | |
| 1H3 + 2G4 (1:1) | 0/4 | 0% | 11.8 ± 3.9 | 31% |
| (n = 4) | | | | |
| 1H3 + 4G7 (1:1) | 0/4 | 0% | 8.8 ± 0.5 | 20% |
| (n = 4) | | | | |
| 1H3 + 2G4 (1:2) | 1/4 | 25% | 10 ± 1 | 14% |
| (n = 4) | | | | |
| 1H3 + 4G7 (1:2) | 0/4 | 0% | 8.5 ± 1.0 | 23% |
| (n = 4) | | | | |

Based on clinical scores, the Group F animals in Table 4 did not appear to be as sick as animals E4 and E6, both of whom were near the clinical limit for IACUC mandated euthanasia at 5 and 7 dpi, respectively. Animal E4 had a flushed face and severe rash on more than 40% of its body surface between 5 to 8 dpi in addition to nasal haemorrhage at 7 dpi, whereas animal E6 had a flushed face and petechiae on its arms and legs between 7 to 9 dpi, in addition to jaundice between 10 to 14 dpi. This indicates that host genetic factors may play a role in the differential susceptibility of individual NHPs to EBOV-K infections. Fever, leukocytosis, thrombocytopenia, and a severe rash symptomatic of EBOV disease progression was detected in both E4 and E6 (Table 4). Increases in the level of liver enzymes ALT (10- to 30-fold increase), ALP (2- to 3-fold), and total bilirubin (TBIL, 3- to 11-fold) indicate significant liver damage, a hallmark of filovirus infections. However, ZMAPP was successful in reversing observed disease symptoms and physiological abnormalities after 12 dpi, 2 days after the last ZMAPP administration (Table 4). Furthermore, ZMAPP treatment was able to lower the high virus loads observed in animals F2 and F5 (up to 10⁶ TCID₅₀/ml) to undetectable levels by 14 dpi.

### Example 4. ZMAPP Cross-Reacts with Guinea EBOV

While the results were very promising with EBOV-K infected NHPs, it was unknown whether therapy with ZMAPP would be similarly effective against the Guinean variant of EBOV (EBOV-G), the virus responsible for the West African outbreak. Direct comparison of published amino acid sequences between EBOV-G and EBOV-K showed that the epitopes targeted by ZMAPP (38) (39) were not mutated between the two virus variants, suggesting that the antibodies should retain their specificity for the viral glycoprotein. To confirm this, in vitro assays were carried out to compare the binding affinity of c13C6, c2G4 and c4G7 to sucrose-purified EBOV-G and EBOV-K. As measured by ELISA, the ZMAPP components showed slightly better binding kinetics for EBOV-G than for EBOV-K. Additionally, the neutralizing activity of individual mAbs was evaluated in the absence of complement for c2G4 and c4G7, and in the presence of complement for c13C6, as they have previously been shown to neutralize EBOV only under this condition(28). The results supported the ELISA binding data, with comparable neutralizing activities between the two viruses.

### Example 5. Compassionate Use of ZMAPP on Patients Infected with Ebola

### Study Participants

All patients had a confirmed positive PCR test for Ebola virus prior to administration of the mAbs. Local care givers were responsible for patient selection. Other than symptoms consistent with EVD and virologic diagnosis, the criteria included adult age, severity of symptoms, stage of disease, status as health care workers in an environment critically lacking such personnel, absence of other therapeutic options, patient acceptance of the risk, and drug availability. The proximity of product supply also played a role in patient selection. ZMAPP that was being stored in Africa was used to initiate treatment of the first two patients, and additional doses that had been pre-positioned in the EU under the regulatory authority of SwissMedic were used to treat the third and seventh patients.

Patients received supportive care and additional clinical testing according to the standards and practices of their treating institutions. As such, these measures were not consistent across all patients. Telephonic consultations with prior investigators were incorporated into the preparation for each new patient exposure.

### Study Procedures

At time of use, ZMAPP vials were thawed and diluted in normal saline or Ringers Lactate to a concentration of 4 mg/mL. The prepared solution was either pre-filtered under aseptic conditions through a 0.2 μm, low-protein binding filter, or administered with an in-line 0.2 μm filter. The recommended treatment plan was three doses of 50 mg/kg at three day intervals (i.e., Day 1, Day 4 and Day 7) via intravenous (IV) infusion. For the first infusion, the recommended starting infusion rate was 50 mg/hour (12.5 mL), escalating by 50 mg/hr every 30 minutes up to a maximum rate of 400 mg/hr. Provided that the first infusion was well tolerated, the second and third infusions had a recommended starting at rate of 200 mg/hr, escalating by 200 mg/hr every 15-30 minutes up to a maximum rate of 800 mg/hr. The total duration of infusion ranged from 5 to 20 hours per dose.

All patients were pre-medicated with an antihistamine (diphenhydramine, promethazine or chlorphenamine) prior to receiving each dose of ZMAPP. Administration of these agents was continued at the physicians' discretion during administration. Antipyretics were administered as needed for patient comfort.

Viral load was assessed by quantitative real time reverse transcriptase polymerase chain reaction (qRT-PCR). These assays amplify and detect both positive and negative strand RNA sequences, and do not distinguish between mRNA and viral genomic RNA. For patients 1 & 2 nucleic acid was extracted from 100 μL of undiluted plasma using the Magmax Pathogen RNA/DNA kit (Life Technologies). A qRT-PCR assay targeting the nucleoprotein gene of Ebola virus was used to amplify viral RNA. For patients 3 and 7, nucleic acid amplification tests for detection of EBOV and for quantification of viral load were performed with the use of commercially available kit (Altona; Hamburg, Germany). Standard dilutions were kindly provided by Altona.

Samples were collected and tested during treatment of patients at Emory University Hospital, Royal Free Hospital and Hospital La Paz. Viral load testing protocols for samples collected at Emory University Hospital were conducted by the US Centers for Disease Control (Atlanta, USA) and have been described previously (7). Viral load testing on samples collected at Hospital Universitario La Paz was performed by ISCIII (Madrid, Spain) as described previously (Kreuels B, Wichmann D, Emmerich P, et al. A case of severe Ebola virus infection complicated by gram-negative septicemia. N Engl J Med. 2014. DOI: 0.1056/NEJMoa1411677). Viral load testing of Royal Free samples was performed by Public Health England at the Rare and Imported Pathogens Laboratory (Porton Down, UK). The qRT-PCR assay performed on samples from patients 1 and 2 did not include a concurrently run positive controls to construct a standard curve for precise quantification of RNA copy number. Consequently, Cycle threshold (Ct) values are presented rather than viral RNA copy number. Ct values reflect the number of PCR cycles required to detect the presence of the target sequence with higher Ct values indicating a lower viral load. Samples were considered to be below the assay's limit of detection at a Ct value of >40.

### Viral Load Data

Viral load data are summarized in Table 6. Note that, as these data were generated by different laboratories using different laboratory protocols, the results should not be compared across patients. However, the results do provide a relative indication of changes in viral load within each patient.

TABLE 6
qRT-PCR Results

| | Dose 1 | | Dose 2 | | Dose 3 | |
|---|---|---|---|---|---|---|
| Patient # | Pre | Post | Pre | Post | Pre | Post |
| 1 | — | — | Ct = 26 | Ct = 31.1 | Ct = 32.8 | Ct = 34.8 |
| 2 | — | — | — | — | Ct = 34.9 | Ct = 36 |
| 3 | 1.5 × 10⁶ copies/mL | 2.3 × 10⁵ copies/mL | — | — | — | — |
| 7 | 1.5 × 10⁶ copies/mL | 3.0 × 10⁴ copies/mL | BLOD | BLOD | — | — |

BLOD—Below Limit of Detection

Prior to administration of dose 2, patient 1 had a Ct value of 26. One day after dose 2, the Ct value was 31.1, an ˜32-fold reduction in serum viral RNA. For Patient 2, the Ct values for samples collected before and one day after administration of dose 3 were 34.9 and 36, respectively. The only earlier data available from these patients were collected from samples that preceded ZMAPP administration, and were generated by a different laboratory and protocol. Therefore, those data are not included herein to avoid presenting a false baseline.

Patient 3 had a viral load of 1.5×106 copies/mL serum immediately prior to administration of dose 1. Viral load was 3.6×106 copies/mL serum in the sample collected immediately after administration of this dose, and declined to 2.3×105 copies/mL serum one day after administration of dose 1. Patient 7 had a viral load of 1.5×106 copies/mL serum prior to administration of dose 1. One day after administration, the viral load for this patient was determined to be 3.0×104 copies/mL. Viral load progressively declined below the assay limit of detection immediately prior to administration of dose 2.

### Patient Outcome

Patient outcomes are summarized in Table 7. Of the seven patients who received ZMAPP, five were alive at the time of discharge and two died while hospitalized. Patients who survived were discharged 15-30 days after symptom onset. Patients 3 and 6 died 12 and 26 days after symptom onset, respectively.

TABLE 7
Patient Outcome

| | Number of days | | | | |
|---|---|---|---|---|---|
| | after symptom | | | | |
| | onset before | | Outcome of | | Criteria for discharge |
| Patient | ZMapp | | hospital- | | and sequelae/Cause of |
| # | administration | | ization | doi | death |
| 1 | 9 | doi | Alive at | 30 | Asymptomatic and PCR- |
| | | | discharge | | negative for two |
| | | | | | consecutive days. No |
| | | | | | significant sequelae. |
| 2 | 10 | doi | Alive at | 29 | Asymptomatic and PCR- |
| | | | discharge | | negative for two |
| | | | | | consecutive days. |
| | | | | | Sequelae restricted |
| | | | | | to a mild peripheral |
| | | | | | sensory neuropathy |
| | | | | | without motor |
| | | | | | involvement. |
| 3 | 9 | doi | Death | 12 | Multiple organ failure |
| | | | | | with respiratory |
| | | | | | distress and severe |
| | | | | | shock attributed to |
| | | | | | progression of EVD |
| 4 | 9 | doi | Alive at | 19 | PCR negative and |
| | | | discharge | | asymptomatic for 24 |
| | | | | | hours. No significant |
| | | | | | sequelae. |
| 5 | 12 | doi | Alive at | 25 | PCR negative and |
| | | | discharge | | asymptomatic for 24 |
| | | | | | hours. No significant |
| | | | | | sequelae. |
| 6 | 16 | doi | Death | 26 | Progressive |
| | | | | | neurological and |
| | | | | | cognitive impairments |
| | | | | | including |
| | | | | | disorientation, |
| | | | | | depression and rapid |
| | | | | | onset of stupor |
| | | | | | attributed to |
| | | | | | progression of EVD |
| 7 | 6 | doi | Alive at | 15 | Asymptomatic and blood |
| | | | discharge | | PCR-negative for six |
| | | | | | consecutive days. No |
| | | | | | significant sequelae. |

doi = day of illness/date of symptom onset. This is estimated to be 5 days post infection.

Transparent communication of the results from the use of various therapeutic options is critical to developing strategies for treating patients with EVD. ZMAPP has now been safely administered to seven patients following the dosing scheme proven effective in the macaque model.

Importantly, most of this clinical effort (three full courses and two partial courses to five patients) was conducted in West Africa, demonstrating that the product can be considered for use “in the field”. Whereas sophisticated patient monitoring and laboratory capabilities may not be necessary for safe administration, more refined and/or controlled clinical protocols will require a substantial investment in medical and logistical infrastructure in order to provide proof of benefit. The absence of control data and the sparse qRT-PCR data collected in this case series precludes drawing any conclusions about pharmacologic effect. The reductions in viral load from pre- to post-dose in patients 1 (dose 2), 3 (dose 1) and 7 (dose 1) are suggestive, but could also have been influenced by the patients' own immune responses. Sample collection from patients during treatment in Africa was either extremely sparse or not done at all due to the lack of relevant testing equipment and infection control concerns. Immediate viral load testing would permit testing of alternative treatment schemes, including adaptive designs. Early cessation of treatment after achieving blood PCR negativity (as done in patient 7) could significantly reduce the required dosage in light of the supply limitations for this investigational product.

The data that have been reported regarding the use of this monoclonal antibody combination in non-human primate models have been encouraging (see above). However, while Ebola virus infection in NHPs is known to produce a disease with symptoms similar to those in humans, there are clear differences in the experimental system, including nearly universal mortality in the NHPs. The administration of the viral challenge in the NHP experiments was by intramuscular injection of 4,000×the tissue culture infectious dose 50% (TCID50), which probably results in a more rapid disease progression than occurs during a natural infection in humans. Counterbalancing this, initiation of mAb therapy in the reported patients occurred later in the disease course (6-16 days after onset of frank symptoms) than has been explored in NHP studies, where treatment was initiated up to 5 days post-infection, approximately the date of symptom onset.

When the data from compassionate treatment of human patients is combined with the NHP patient data, it is evident that ZMAPP treatment confers superior survival to infected patients. Preferably, treatment with ZMAPP confers survival rates of at least 70%, more preferably survival rates of at least 75% and even more preferably survival rates of 80% or greater when administered at least five days post infection. Survival rates are also impacted by the time of Administration post infection. For example, administration of ZMAPP as much as 14 days post infection to human patients resulted in survival rates of over 70%. If the ZMAPP therapy were to be administered to such patients at an earlier time point, it is expected that the survival rates would approach those seen in the NHP patient studies.

### Discussion

The West African outbreak of 2014 has highlighted the troubling absence of available vaccine or therapeutic options to save thousands of lives and stop the spread of EBOV. The lack of a clinically acceptable treatment offer limited incentive for people who suspect they might be infected to report themselves for medical help. Several previous studies have showed that antibodies are crucial for host survival from EBOV(40) (41) (42). Prior NHP studies have also demonstrated the ZMAb cocktail could protect 100% or 50% of animals when dosing was initiated 1 or 2 dpi, while the MB-003 cocktail protected 67% of animals with the same dosing regimen. Before the success with mAb-based therapies, other candidate therapeutics had only demonstrated efficacy when given within 60 minutes of EBOV exposure.

Our results with ZMAPP, a cocktail comprising of individual mAbs selected from MB-003 and ZMAb, demonstrate for the first time the successful protection of NHPs from EBOV disease when intervention was initiated as late as 5 dpi. In the preceding ZMapp1/ZMapp2 experiment, 11 of 12 treated animals had detectable fever (with the exception of A4), and live virus could be detected in the blood of 11 of 12 animals (with the exception of A3) by 3 dpi. Therefore, for the majority of these animals, treatment was therapeutic (as opposed to post-exposure prophylaxis), initiated after two detectable triggers of disease. ZMapp2 was able to protect 5 of 6 animals when administered at 3 dpi. For reasons currently unknown, the lone non-survivor (B3) experienced a viremia of 10⁶ TCID₅₀ at 3 dpi, which is 100-fold greater than all other NHPs and approximately 10-fold higher than what ZMAb has been reported to suppress in a previous study(31). This indicates enhanced EBOV replication in this animal, possibly due to host factors. It was important to note that despite the high levels of live circulating virus detected in B3, ZMapp2 administration was still able to prolong the life of this animal to 9 dpi, and suggests that in cases of high viremia such as this, the dosage of mAbs should be increased.

The highlight of these experimental results is undoubtedly ZMAPP, which was able to reverse severe EBOV disease as indicated by the elevated liver enzymes, mucosal hemorrhages and rash in animals E4 and E6. The high viremia (up to 10⁶ TCID₅₀/ml of blood in some animals at the time of intervention) could also be effectively controlled without the presence of escape mutants, leading to full recovery of all treated NHPs by 28 dpi. In the absence of direct evidence demonstrating ZMAPP efficacy against lethal EBOV-G infection in NHPs, results from ELISA and neutralizing antibody assays show that binding specificity is not abrogated between EBOV-K and EBOV-G, and therefore the levels of protection should not be affected. The compassionate use of ZMAPP in two infected American healthcare workers with positive results pertaining to survival and reversion of EBOV disease (43), supports this assertion. Rhesus macaques have approximately 55-80 ml of blood per kg of body weight (44); at a dose of 50 mg/kg of antibodies, the estimated starting concentration is approximately 625-909 μg/ml of blood (total; ˜200-300 μg/ml for each antibody). Therefore, the low EC₅₀ values for EBOV-G (0.004-0.02 μg/ml) bode well for treating EBOV-G infections with ZMAPP.

Since the host antibody response is known to correlate with and is required for protection from EBOV infections (41) (42), mAb-based treatments are likely to form the centerpiece of any future therapeutic strategies for fighting EBOV outbreaks. However, whether ZMAPP-treated survivors can be susceptible to re-infection is unknown. In a previous study of murine ZMAb-treated, EBOV-challenged NHP survivors, a re-challenge of these animals with the same virus at 10 and 13 weeks after initial challenge yielded 6 of 6 survivors and 4 of 6 survivors, respectively (45). While specific CD4⁺ and CD8⁺ T-cell responses could be detected in all animals, the circulating levels of glycoprotein (GP)-specific IgG were shown to be 10-fold lower in non-survivors compared to survivors, suggesting that antibody levels may be indicative of protective immunity (45). Sustained immunity with experimental EBOV vaccines in NHPs remain unknown, however in a recent study, a decrease in GP-specific IgG levels due to old age or a suboptimal reaction to the VSVΔG/EBOVGP vaccine in rodents also appear to be indicative of non-survival (46).

ZMAPP consists of a cocktail of highly purified mAbs; which constitutes a less controversial alternative than whole blood transfusions from convalescent survivors, as was performed during the 1995 EBOV outbreak in Kikwit (47). The safety of mAb therapy is well-documented, with generally low rates of adverse reactions, the capacity to confer rapid and specific immunity in all populations, including the young, the elderly and the immunocompromised, and if necessary, the ability to provide higher-than-natural levels of immunity compared to vaccinations (48). The evidence presented here suggests that ZMAPP currently offers the best option of the experimental therapeutics currently in development for treating EBOV-infected patients. We hope that initial safety testing in humans will be undertaken soon, preferably within the next few months, in order to enable the compassionate use of ZMAPP as soon as possible.

In sum, when comparing antibody cocktails that bind to multiple epitopes on the Ebola virus, the most important component of those cocktails in order to achieve complete reversion from lethal Ebola infections in non-human primates is the 13C6 mAb. For example, a cocktail of mAbs consisting of 1H3, 2G4, 4G7 (ZMab (4)), when administered to non-human primates at 48 hours post Ebola infection (EBOV strain Kikwit 95), resulted in a survival rate of 50%. In contrast, the cocktail containing 13C6 (13C6, 2G4, 4G7, ZMapp) when administered to non-human primates up to 5 days post Ebola infection, resulted in 100% survival during the entire course of the study up to 28 days post infection. From these results it can be concluded that the 13C6 mAb contributed an essential binding function that resulted in a survival rate far in excess of the mAb cocktail without 13C6. When compared at equal lower doses (5 mg) in guinea pigs, ZMab resulted in 17% survival whereas ZMAPP resulted in 67% survival (Table 2). Thus, cocktails containing 13C6 are superior other known cocktails or individual monoclonal antibodies, and ZMAPP in particular is vastly more efficacious than other known cocktails for the treatment of Ebola infection.

### REFERENCES

1. Pettitt J, et al. (2013) Therapeutic intervention of Ebola virus infection in rhesus macaques with the MB-003 monoclonal antibody cocktail. Science translational medicine 5(199):199ra113.
2. Qiu X, et al. (2013) Sustained protection against Ebola virus infection following treatment of infected nonhuman primates with ZMAb. Scientific reports 3:3365.
3. Qiu X, et al. (2014) Reversion of advanced Ebola virus disease in nonhuman primates with ZMapp. Nature 514(7520):47-53.
4. Qiu X, et al. (2012) Successful treatment of ebola virus-infected cynomolgus macaques with monoclonal antibodies. Science translational medicine 4(138):138ra181.
5. Hiatt A, Zeitlin L, & Whaley K J (2013) Multiantibody strategies for HIV. Clinical & developmental immunology 2013:632893.
6. Whaley K J, et al. (2012) Emerging Antibody-based Products. Current topics in microbiology and immunology.
7. Bendandi M, et al. (2010) Rapid, high-yield production in plants of individualized idiotype vaccines for non-Hodgkin's lymphoma. Annals of oncology: official journal of the European Society for Medical Oncology/ESMO 21(12):2420-2427.
8. Castilho A & Steinkellner H (2012) Glyco-engineering in plants to produce human-like N-glycan structures. Biotechnology journal 7(9):1088-1098.
9. Loos A, et al. (2014) Expression and glycoengineering of functionally active heteromultimeric IgM in plants. Proceedings of the National Academy of Sciences of the United States of America.
10. Hiatt A (2014) Designed IgM from glycoengineering. Proceedings of the National Academy of Sciences of the United States of America.
11. Schneider J D, et al. (2014) Oligomerization status influences subcellular deposition and glycosylation of recombinant butyrylcholinesterase in Nicotiana benthamiana. Plant biotechnology journal.
12. Schneider J D, et al. (2014) Expression of human butyrylcholinesterase with an engineered glycosylation profile resembling the plasma-derived orthologue. Biotechnology journal 9(4):501-510.
13. Hiatt A & Pauly M (2006) Monoclonal antibodies from plants: a new speed record. Proceedings of the National Academy of Sciences of the United States of America 103(40):14645-14646.
14. Castilho A, et al. (2010) In planta protein sialylation through overexpression of the respective mammalian pathway. The Journal of biological chemistry 285(21):15923-15930.
15. Guile G R, Rudd P M, Wing D R, Prime S B, & Dwek R A (1996) A rapid high-resolution high-performance liquid chromatographic method for separating glycan mixtures and analyzing oligosaccharide profiles. Analytical biochemistry 240(2):210-226.
16. Bausch D G, Sprecher A G, Jeffs B, & Boumandouki P (2008) Treatment of Marburg and Ebola hemorrhagic fevers: a strategy for testing new drugs and vaccines under outbreak conditions. Antiviral Res 78(1):150-161.
17. Baize S, et al. (2014) Emergence of Zaire Ebola Virus Disease in Guinea—Preliminary Report. N Engl J Med.
18. WHO.int (2014) WHO—Ebola virus disease (EVD).
19. CDC.gov (2014) Chronology of Ebola Hemorrhagic Fever Outbreaks.
20. Reliefweb.int (2014) W. African Ebola epidemic ‘likely to last months’: UN.
21. Clark D V, Jahrling P B, & Lawler J V (2012) Clinical management of filovirus-infected patients. Viruses 4(9):1668-1686.
22. Guimard Y, et al. (1999) Organization of patient care during the Ebola hemorrhagic fever epidemic in Kikwit, Democratic Republic of the Congo, 1995. J Infect Dis 179 Suppl 1:S268-273.
23. Hensley L E, et al. (2007) Recombinant human activated protein C for the postexposure treatment of Ebola hemorrhagic fever. J Infect Dis 196 Suppl 2:S390-399.
24. Geisbert T W, et al. (2003) Treatment of Ebola virus infection with a recombinant inhibitor of factor VIIa/tissue factor: a study in rhesus monkeys. Lancet 362(9400):1953-1958.
25. Geisbert T W, et al. (2010) Postexposure protection of non-human primates against a lethal Ebola virus challenge with RNA interference: a proof-of-concept study. Lancet 375(9729):1896-1905.
26. Warren T K, et al. (2010) Advanced antisense therapies for postexposure protection against lethal filovirus infections. Nat Med 16(9):991-994.
27. Feldmann H, et al. (2007) Effective post-exposure treatment of Ebola infection. PLoS Pathog 3(1):e2.
28. Olinger G G, Jr., et al. (2012) Delayed treatment of Ebola virus infection with plant-derived monoclonal antibodies provides protection in rhesus macaques. Proc Natl Acad Sci USA 109(44):18030-18035.
29. Qiu X, et al. (2012) Successful treatment of ebola virus-infected cynomolgus macaques with monoclonal antibodies. Sci Transl Med 4(138):138ra181.
30. Pettitt J, et al. (2013) Therapeutic intervention of Ebola virus infection in rhesus macaques with the MB-003 monoclonal antibody cocktail. Sci Transl Med 5(199):199ra113.
31. Qiu X, et al. (2013) mAbs and Ad-Vectored IFN-alpha therapy rescue ebola-infected nonhuman primates when administered after the detection of viremia and symptoms. Sci Transl Med 5(207):207ra143.
32. Giritch A, et al. (2006) Rapid high-yield expression of full-size IgG antibodies in plants coinfected with noncompeting viral vectors. Proc Natl Acad Sci USA 103(40):14701-14706.
33. Zeitlin L, et al. (2011) Enhanced potency of a fucose-free monoclonal antibody being developed as an Ebola virus immunoprotectant. Proc Natl Acad Sci USA 108(51):20690-20694.
34. Jahrling P B, et al. (1999) Evaluation of immune globulin and recombinant interferon-alpha2b for treatment of experimental Ebola virus infections. J Infect Dis 179 Suppl 1:S224-234.
35. Connolly B M, et al. (1999) Pathogenesis of experimental Ebola virus infection in guinea pigs. J Infect Dis 179 Suppl 1:S203-217.
36. Reed L J & Muench H (1938) A simple method of estimating fifty percent endpoints. American journal of hygiene 27:493-497.
37. Qiu X, et al. (2012) Ebola GP-specific monoclonal antibodies protect mice and guinea pigs from lethal Ebola virus infection. PLoS Negl Trop Dis 6(3):e1575.
38. Wilson J A, et al. (2000) Epitopes involved in antibody-mediated protection from Ebola virus. Science 287(5458):1664-1666.
39. Qiu X, et al. (2011) Characterization of Zaire ebolavirus glycoprotein-specific monoclonal antibodies. Clin Immunol 141(2):218-227.
40. Dye J M, et al. (2012) Postexposure antibody prophylaxis protects nonhuman primates from filovirus disease. Proc Natl Acad Sci USA 109(13):5034-5039.
41. Wong G, et al. (2012) Immune parameters correlate with protection against ebola virus infection in rodents and nonhuman primates. Sci Transl Med 4(158):158ra146.
42. Marzi A, et al. (2013) Antibodies are necessary for rVSV/ZEBOV-GP-mediated protection against lethal Ebola virus challenge in nonhuman primates. Proc Natl Acad Sci USA 110(5):1893-1898.
43. Promedmail.org (2014) EBOLA VIRUS DISEASE—WEST AFRICA (117): WHO, NIGERIA, LIBERIA, DRUG, MORE.
44. NC3RS.org (2014) Practical blood sample volumes for laboratory animals, domestic species and non-human primates.
45. Qiu X, et al. (2013) Sustained protection against Ebola virus infection following treatment of infected nonhuman primates with ZMAb. Sci Rep 3:3365.
46. Wong G, et al. (2014) Immunization with Vesicular Stomatitis Virus vaccine expressing the Ebola glycoprotein provides sustained long-term protection in rodents. Vaccine in press.
47. Mupapa K, et al. (1999) Treatment of Ebola hemorrhagic fever with blood transfusions from convalescent patients. International Scientific and Technical Committee. J Infect Dis 179 Suppl 1:S18-23.
48. UPMChealthsecurity.org (2013) Next-Generation Monoclonal Antibodies: Challenges and Opportunities. (UPMC Center for Biosecurity).

We claim:

1. A composition for the treatment of Ebola, the composition comprising:
a therapeutically effective combination of
i. a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof;
ii. a second monoclonal antibody comprising a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof; and
iii. a third monoclonal antibody comprising a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

2. The composition of claim 1, further comprising: a pharmaceutically acceptable excipient or carrier.

3. The composition of claim 1, wherein at least one of the first, second, and third monoclonal antibodies comprise a predominantly single glycoform.

4. The composition of claim 3, wherein the predominantly single glycoform comprises the GnGn glycan.

5. The composition of claim 3, wherein the predominantly single glycoform comprises galactosylated glycans.

6. The composition of claim 3, wherein the predominantly single glycoform comprises sialylated glycans.

7. The composition of claim 3, wherein the predominantly single glycoform comprises less than 5% fucose or xylose.

8. A composition for the treatment of Ebola, the composition comprising:
a therapeutically effective combination of
i. a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof; and
ii. a second monoclonal antibody that binds the Ebola glycoprotein;
iii. wherein administration of the composition to patients five days following infection with the Ebola virus results in at least a 70% survival rate.

9. The composition of claim 8, wherein the second monoclonal antibody comprises a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof.

10. The composition of claim 8, wherein the second monoclonal antibody comprises a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

11. The composition of claim 8, wherein the patient is a human.

12. The composition of claim 8, further comprising: a pharmaceutically acceptable excipient or carrier.

13. The composition of claim 8, wherein at least one of the first, and second monoclonal antibodies comprise a predominantly single glycoform.

14. The composition of claim 13, wherein the predominantly single glycoform comprises the GnGn glycan.

15. The composition of claim 13, wherein the predominantly single glycoform comprises galactosylated glycans.

16. The composition of claim 13, wherein the predominantly single glycoform comprises sialylated glycans.

17. The composition of claim 13, wherein the predominantly single glycoform comprises less than 5% fucose or xylose.

18. A method for treating Ebola infection in a primate, the method comprising:
i. identifying a primate in need of Ebola treatment; and
ii. administering to the patient a therapeutically effective amount of a composition comprising a combination of:
a) a first monoclonal antibody comprising a light chain variable region comprising an amino acid sequence deduced from the nucleic acid molecule as set forth in SEQ ID NO: 4, and humanized variants thereof, and a heavy chain variable region comprising an amino acid sequence deduced from the nucleic acid molecule as set forth in SEQ. ID NO: 3, and humanized variants thereof; and
b) a second monoclonal antibody; wherein said second monoclonal antibody is selected from a group consisting of: 1) a monoclonal antibody comprising a light chain variable region comprising an amino acid sequence deduced from the nucleic acid molecule as set forth in SEQ ID NO: 6, and humanized variants thereof, and a heavy chain variable region comprising an amino acid sequence deduced from the nucleic acid molecule as set forth in SEQ. ID NO: 5, and humanized variants thereof; and 2) a monoclonal antibody comprising a light chain variable region comprising an amino acid sequence deduced from the nucleic acid molecule as set forth in SEQ ID NO: 8, and humanized variants thereof, and a heavy chain variable region comprising an amino acid sequence deduced from the nucleic acid molecule as set forth in SEQ. ID NO: 7, and humanized variants thereof.

19. The method of claim 18, wherein the primate is a human.

20. The method of claim 18, wherein the therapeutically effective composition further comprises a pharmaceutically acceptable excipient or carrier.

21. The composition of claim 18, wherein at least one of the first, and second monoclonal antibodies comprise a predominantly single glycoform.

22. The composition of claim 21, wherein the predominantly single glycoform comprises the GnGn glycan.

23. The composition of claim 21, wherein the predominantly single glycoform comprises galactosylated glycans.

24. The composition of claim 21, wherein the predominantly single glycoform comprises sialylated glycans.

25. The composition of claim 13, wherein the predominantly single glycoform comprises less than 5% fucose or xylose.


SEQUENCE LISTING (25 sequences)

SEQ ID NO: 1 - protein, 122 aa, Mus musculus
QLTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSS

SEQ ID NO: 2 - protein, 107 aa, Mus musculus
DIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKLELR

SEQ ID NO: 3 - DNA, 366 bp, Mus musculus
cagcttactttgaaagagtccggtccaggaatccttaagccttctcagactttgtctctcacctgttctttgtcaggattctctctttccacttctggagttggagttggttggttcagacaaccttctggaaagggtcttgagtggcttgctctcatctggtgggacgatgacaagtactacaacccaagcttgaagtctcagttgtctatctccaaggacttctctaggaaccaggtgttcttgaagatctccaatgtggacattgccgataccgctacttactattgcgccagaagggacccattcggttacgacaacgctatgggatactggggtcaaggaacttctgtgactgtttcctca

SEQ ID NO: 4 - DNA, 321 bp, Mus musculus
gatattgtgatgacccagagccaaaagttcatgtccacctctgttggtgatagagtgtcacttacttgcaaggcttcccagaatgtgggaactgctgttgcctggtatcaacagaagccaggtcagtctcctaagttgcttatctactcagctagcaaccgttacactggagttccagaccgtttcactggttctggatccggtacagatttcacccttacaatctccaacatgcagtcagaggacttggcagattacttctgccagcagtactcttcctaccctcttacttttggtgcaggaactaagcttgagctcaga

SEQ ID NO: 5 - DNA, 364 bp, Mus musculus
tggaggaggcttgatgcaacctggaggatccatgaaactctcctgtgttgcctcaggattcactttcagtaactactggatgaactgggtccgccagtctccagagaaggggcttgagtgggttgctgaaattagattgaaatctaataattatgcaacacattatgcggagtctgtgaaagggaggttcaccatttcaagagatgattccaaaaggagtgtctacctgcaaatgaataccttaagagctgaagacactggcatttattactgtacccgggggaatggtaactacagggctatggactactggggtcaaggaacctcagtcaccgtctcctcagccaaaacaacacccccatca

SEQ ID NO: 6 - DNA, 306 bp, Mus musculus
gcctccctatctgtatctgtgggagaaactgtctccatcacatgtcgagcaagtgagaatatttacagtagtttagcatggtatcagcagaaacagggaaaatctcctcagctcctggtctattctgcaacaatcttagcagatggtgtgccatcaaggttcagtggcagtggatcaggcactcagtattccctcaagatcaacagcctgcagtctgaagattttgggacttattactgtcaacatttttggggtactccgtacacgttcggaggggggaccaagctggaaataaaacgggctgat

SEQ ID NO: 7 - DNA, 358 bp, Mus musculus
tggacctgagctggagatgcctggcgcttcagtgaagatatcctgcaaggcttctggttcctcattcactggcttcagtatgaactgggtgaagcagagcaatggaaagagccttgagtggattggaaatattgatacttattatggtggtactacctacaaccagaaattcaagggcaaggccacattgactgtggacaaatcctccagcacagcctacatgcagctcaagagcctgacatctgaggactctgcagtctattactgtgcaagatcggcctactacggtagtacttttgcttactggggccaagggactctggtcactgtctctgcagccaaaacaacagccccatcg

SEQ ID NO: 8 - DNA, 306 bp, Mus musculus
gcctccctatctgcatctgtgggagaaactgtcaccatcacatgtcgagcaagtgagaatatttacagttatttagcatggtatcagcagaaacagggaaaatctcctcagctcctggtctataatgccaaaaccttaatagagggtgtgccatcaaggttcagtggcagtggatcaggcacacagttttctctgaagatcaacagcctgcagcctgaagattttgggagttatttctgtcaacatcattttggtactccattcacattcggctcggggacagagttggaaataaaacgggctgat

SEQ ID NO: 9 - protein, 11 aa, Mus musculus
KASQNVGTAVA

SEQ ID NO: 10 - protein, 7 aa, Mus musculus
SASNRYT

SEQ ID NO: 11 - protein, 9 aa, Mus musculus
QQYSSYPLT

SEQ ID NO: 12 - protein, 12 aa, Mus musculus
GFSLSTSGVGVG

SEQ ID NO: 13 - protein, 14 aa, Mus musculus
LIWWDDDKYYNPSL

SEQ ID NO: 14 - protein, 12 aa, Mus musculus
RDPFGYDNAMGY

SEQ ID NO: 15 - protein, 25 aa, Homo sapiens
QVQLKESGPGLLKPSQTLSLTCTVS

SEQ ID NO: 16 - protein, 14 aa, Homo sapiens
WIRQPAGKGLEWIA

SEQ ID NO: 17 - protein, 34 aa, Homo sapiens
KSRLTITKDTSKNQVVLTMTNMDPVDTATYYCAR

SEQ ID NO: 18 - protein, 23 aa, Homo sapiens
DIQMTQSPSFLSASVGDRVTITC

SEQ ID NO: 19 - protein, 15 aa, Homo sapiens
WYQQKPGQPPKLLIY

SEQ ID NO: 20 - protein, 32 aa, Homo sapiens
GVPDRFSGSGSGTDFTLTISSLQAEDVAVYYC

SEQ ID NO: 21 - protein, 25 aa, Homo sapiens
QVQLLESGPGILKPSQTLSLTCSLS

SEQ ID NO: 22 - protein, 25 aa, Homo sapiens
QVQLLESGGGVVKPGQTLSLTCSLS

SEQ ID NO: 23 - protein, 25 aa, Homo sapiens
DVKLLESGGGLVQPGGSLKLSCAAS

SEQ ID NO: 24 - protein, 23 aa, Homo sapiens
DIVMTQSPLSLSTSVGDRVSLTC

SEQ ID NO: 25 - protein, 23 aa, Homo sapiens
DVLLTQIPLSLSTSVGDRVSLTC